(19) United States
(12) Patent Application Publication
(10) Pub. No.: US 2019/0391215 A1
(43) Pub. Date: Dec. 26, 2019

(54) ATOMIC MAGNETOMETER, GRADIOMETER, AND BIOMAGNETISM MEASUREMENT APPARATUS

(71) Applicant: TAKANOBU OSAKA, Ishikawa (JP)

(72) Inventor: TAKANOBU OSAKA, Ishikawa (JP)

(73) Assignee: Ricoh Company, Ltd., Tokyo (JP)

(21) Appl. No.: 16/447,089

(22) Filed: Jun. 20, 2019

(30) Foreign Application Priority Data
Jun. 26, 2018 (JP) 2018-121219

(51) Int. Cl. G01R 33/26; G01R 33/022; A61B 5/04

(52) U.S. Cl. CPC G01R 33/26; A61B 5/04007; G01R 33/022

(57) ABSTRACT

An atomic magnetometer includes a laser light source that emits light, a light splitting unit that splits the light emitted from the laser light source into a first light beam and a second light beam, a transparent cell filled with an alkali metal atom and through which the first light beam is transmitted; and a photodetector.

### CROSS-REFERENCE TO RELATED APPLICATIONS

This patent application is based on and claims priority pursuant to 35 U.S.C. § 119(a) to Japanese Patent Application No. 2018-121219, filed on Jun. 26, 2018, in the Japan Patent Office, the entire disclosure of which is hereby incorporated by reference herein.

### BACKGROUND

### Technical Field

The present invention relates to an atomic magnetometer, a gradiometer, and a biomagnetism measurement apparatus.

### Discussion of the Background Art

A magnetic field emitted from a living body is called biomagnetism. As an apparatus which measures weak biomagnetism, a magnetoencephalography (MEG) which measures a magnetic field (brain magnetic field) generated by an electrical activity of a brain nerve cell has been known.

Further, as a magnetic sensor used in the magnetoencephalography (MEG), an optical pumping atomic magnetometer which does not require a cryogenic environment has been known.

For optical pumping atomic magnetometer, a technology, in which a photodetector receives P waves and S waves of probe light having passed through a transparent cell, respectively, and a magnitude of a magnetic field is detected from a rotation angle of a polarization plane corresponding to a difference between the P waves and the S waves, has been disclosed.

### SUMMARY

Example embodiments of the present invention include an atomic magnetometer including: a laser light source that emits light; a light splitting unit that splits the light emitted from the laser light source into at least a first light beam and a second light beam; a transparent cell filled with an alkali metal atom and through which the first light beam is transmitted; and a photodetector that receives the first light beam which has transmitted through the cell and the second light beam which has not transmitted through the cell.

Example embodiments of the present invention include: an atomic magnetometer for measuring a strength of a magnetic field using probe light, the atomic magnetometer including: a laser light source that emits probe light; a light splitting unit that splits the probe light into a first light beam and a second light beam; a transparent cell filled with an alkali metal atom and through which the first light beam is transmitted; and a photodetector that detects an intensity of interfering light between the first light beam having transmitted through the cell and the second light beam.

### BRIEF DESCRIPTION OF THE DRAWINGS

A more complete appreciation of the disclosure and many of the attendant advantages and features thereof can be readily obtained and understood from the following detailed description with reference to the accompanying drawings, wherein:

FIG. 1 is a view for describing a configuration of an atomic magnetometer according to a first embodiment;

FIGS. 2A and 2B are views for describing a configuration of a light splitting unit according to the embodiment;

FIGS. 3A to 3D are views for describing a relation between first light and second light according to the embodiment;

FIGS. 4A and 4B are views for describing behaviors of the first light beam and the second light beam according to the embodiment;

FIGS. 5A and 5B are views for describing another example of the behaviors of the first light and the second light according to the embodiment;

FIG. 6 is a view for describing a configuration of the atomic magnetometer in a case of applying a predetermined phase difference according to the embodiment;

FIG. 7 is a view for describing a configuration of the atomic magnetometer in a case where one polarizer is included according to the embodiment;

FIG. 8 is a view for describing a configuration of the atomic magnetometer in a case where one ½ wave plate is included according to the embodiment;

FIGS. 9A to 9D are views illustrating simulation results of an interference fringe pattern according to the embodiment;

FIG. 10 is a view for describing a configuration for adjusting a position of the photodetector according to the embodiment;

FIG. 11 is a view for describing an opening of the photodetector according to the embodiment;

FIG. 12 is a view for describing a photodetector array including a plurality of pixels according to the embodiment;

FIG. 13 is a view for describing a configuration of an atomic magnetometer according to a second embodiment;

FIG. 14 is a view for describing a configuration of an atomic magnetometer according to a third embodiment;

FIG. 15 is a view for describing a configuration of a gradiometer according to a fourth embodiment;

FIG. 16 is a view for describing a configuration of a biomagnetism measurement apparatus according to a fifth embodiment;

FIG. 17 is a functional block diagram illustrating components of a controller according to the fifth embodiment;

FIG. 18 is a view for describing a configuration of a biomagnetism measurement apparatus according to a first modified example;

FIG. 19 is a view for describing a configuration of a biomagnetism measurement apparatus according to a second modified example; and

FIG. 20 is a view for describing a configuration of a biomagnetism measurement apparatus according to a third modified example.

The accompanying drawings are intended to depict embodiments of the present invention and should not be interpreted to limit the scope thereof. The accompanying drawings are not to be considered as drawn to scale unless explicitly noted.

### DETAILED DESCRIPTION

The terminology used herein is for the purpose of describing particular embodiments only and is not intended to be limiting of the present invention. As used herein, the singular forms “a”, “an” and “the” are intended to include the plural forms as well, unless the context clearly indicates otherwise.

In describing embodiments illustrated in the drawings, specific terminology is employed for the sake of clarity. However, the disclosure of this specification is not intended to be limited to the specific terminology so selected and it is to be understood that each specific element includes all technical equivalents that have a similar function, operate in a similar manner, and achieve a similar result.

Hereinafter, embodiments of the present invention will be described with reference to the drawings. In the respective drawings, the same components will be denoted by the same reference numeral, and an overlapping description will be omitted. Further, solid arrows represent directions in the respective drawings, the directions including an X direction, a Y direction, and a Z direction.

An atomic magnetometer according to an embodiment uses spin polarization of an alkali metal atom generated by optical pumping to measure a strength of a magnetic field. Here, the optical pumping is a method for making the numbers of occupying atoms in two adjacent energy levels be greatly different from each other by using light.

The alkali metal atom subjected to the optical pumping is spin-polarized. The magnetic field as a measurement target rotates the polarized spin to rotate a polarization plane of linearly polarized light incident as the probe light. The atomic magnetometer according to the embodiment detects a rotation angle of a polarization plane of probe light to measure a strength of a magnetic field.

### First Embodiment

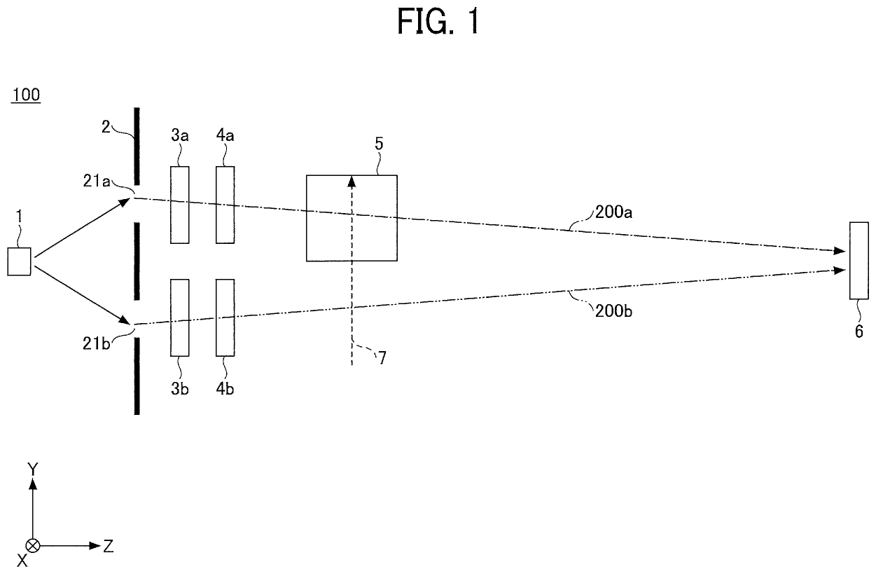

First, a first embodiment will be described. FIG. 1 is a view for describing a configuration of an atomic magnetometer according to the present embodiment.

An atomic magnetometer 100 includes a light source 1, a light splitting unit 2, polarizers 3a and 3b, ½ wave plates 4a and 4b, a cell 5, and a photodetector 6. The cell 5 is a transparent container filled with a vapor of an alkali metal atom. The alkali metal atom may be any one of potassium (K), rubidium (Rb), and cesium (Cs). The cell 5 may be filled with inert gas (buffer gas) which increases a relaxation time of the atom, such as helium, nitrogen, and argon, in addition to the alkali metal atom. Further, the cell 5 may have an inner wall coated with paraffin or the like to prevent relaxation of spin polarization of the atom.

A portion of the cell 5 where light is incident and emitted may be made of a material through which the light can be transmitted, for example, a glass material. A material of portions of the cell 5 other than the portion of the cell 5 where the light is incident and emitted can be a glass material, a metal material, a resin material, or the like, but is not particularly limited thereto. However, the cell 5 may also be entirely manufactured by using a material through which light can be transmitted, such as borosilicate glass.

Pump light 7 indicated by a dotted arrow in FIG. 1 is incident on the cell 5. The pump light 7 is light having an absorption wavelength (for example, 895 nm corresponding to a D1 line of 133 Cs) of the alkali metal atom in the cell 5. As the pump light 7, for example, light emitted from a vertical cavity surface emitting laser (VCSEL) can be used. However, the pump light 7 may be any light having the absorption wavelength of the alkali metal atom and is not limited to the light emitted from the VCSEL.

The pump light 7 may be nearly circularly polarized light. The alkali metal atom is excited by the nearly circularly polarized light, thereby making it possible to increase a pumping rate. In order to obtain circularly polarized light, a ¼ wave plate having a function of converting linearly polarized light into nearly circularly polarized light can be used. For example, the ¼ wave plate may be disposed between a VCSEL emitting linearly polarized light and the cell 5 so that an optical axis is inclined at an angle of 45 degrees with respect to a polarization plane of the linearly polarized light.

The polarization plane is a plane including a traveling direction of light, an electric field, or an oscillation direction of the magnetic field. Since the polarization plane includes the oscillation direction, hereinafter, an oscillation direction of linearly polarized light is referred to as the polarization plane in some cases. Further, the optical axis of the ¼ wave plate is a fast axis or a slow axis of the ¼ wave plate.

In FIG. 1, an example in which the pump light 7 is incident on the cell 5 from a negative Y direction toward a positive Y direction is illustrated, but the present invention is not limited thereto. For example, the pump light 7 may be incident on the cell 5 from the positive Y direction toward the negative Y direction. Further, the pump light 7 may also be incident on the cell 5 from a positive X direction toward a negative X direction, or from the negative X direction toward the positive X direction.

The light source 1 is a laser light source and emits laser light having a wavelength different from the wavelength of the pump light 7. Examples of the light source 1 include a VCSEL, a laser diode (LD), a distributed Bragg reflector (DBR) laser, and the like. The laser light emitted from the light source 1 is an example of the “probe light”.

Examples of the light splitting unit 2 include a pinhole array including two pinholes 21a and 21b. The probe light which is diverging light emitted from the light source 1 is split into two light beams including a light beam passing through the pinhole 21a of the light splitting unit 2 and a light beam passing through the pinhole 21b.

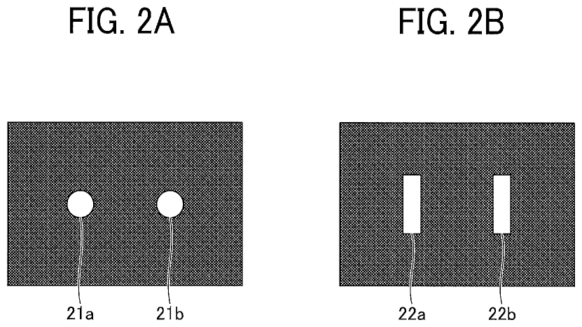

The pinhole array is manufactured, for example, by providing two through holes in a metal flat plate. However, the light splitting unit 2 is not limited thereto, and may also be a slit array. FIGS. 2A and 2B are views for describing a configuration of the light splitting unit 2 according to the embodiment. FIG. 2A is a front view illustrating the pinhole array including the pinholes 21a and 21b. FIG. 2B is a front view illustrating a slit array including slits 22a and 22b. In FIGS. 2A and 2B, a region in black is a region shielding the probe light, and the pinholes 21a and 21b or the slits 22a and 22b, which are regions in white, are regions allowing the probe light to pass.

Referring back to FIG. 1, light having passed through the pinhole 21a passes through the polarizer 3a to be linearly polarized. Alternatively, in a case where linearly polarized light is emitted from the light source 1, the light passes through the polarizer 3a to increase a polarization degree of the linearly polarized light. Examples of the polarizer 3a include a polarizing plate. However, the polarizer 3a is not limited thereto, and a Glan-Thompson prism or the like may be used to obtain linearly polarized light with a higher polarization degree.

The linearly polarized light after passing through the polarizer 3a is incident on the ½ wave plate 4a. The ½ wave plate 4a is an optical element which rotates a polarization plane of the incident linearly polarized light to emit light. For example, in a case of rotating an optical axis of the ½ wave plate 4a by an angle φ about an axis in a traveling direction of the light, linearly polarized light, of which a polarization plane is rotated by an angle 2φ with respect to the polarization plane of the linearly polarized light incident on the ½ wave plate 4a, is emitted from the ½ wave plate 4a.

The light emitted from the ½ wave plate 4a passes through the cell 5 and is incident on the photodetector 6. As indicated by a chain line in FIG. 1, light having passed through the pinhole 21a and incident on the photodetector 6, of the two light beams which have been split by the light splitting unit 2, is a first light beam 200a.

Meanwhile, light having passed through the pinhole 21b passes through the polarizer 3b to be linearly polarized. Alternatively, in a case where linearly polarized light is emitted from the light source 1, the light passes through the polarizer 3a to increase a polarization degree of the linearly polarized light. The linearly polarized light after passing through the polarizer 3b is incident on the ½ wave plate 4b, and a polarization plane of the linearly polarized light is rotated by a predetermined angle to emit the light. The light emitted from the ½ wave plate 4b is directly incident on the photodetector 6 without passing through the cell 5. As indicated by a two point chain line in FIG. 1, the light passing through the pinhole 21b and incident on the photodetector 6, of the two light beams which have been split by the light splitting unit 2, is a second light beam 200b.

Examples of the photodetector 6 include a photodiode which outputs a voltage signal according to an intensity of received light. However, the photodetector 6 is not limited thereto, and a photodiode array or an imaging device such as a metal oxide semiconductor (MOS) device, a complementary metal oxide semiconductor (CMOS) device, or a charge coupled device (CCD) may be used.

An angle between the optical axes of the ½ wave plates 4a and 4b can be adjusted to set an angle α between a polarization plane of the linearly polarized first light beam 200a and a polarization plane of the linearly polarized second light beam 200b to a predetermined angle. As an example, in a case where the angle α is 0 degrees, the polarization plane of the linearly polarized first light beam 200a is in parallel with the polarization plane of the linearly polarized second light beam 200b. In a case where the angle α is 90 degrees, the polarization plane of the linearly polarized first light beam 200a is orthogonal to the polarization plane of the linearly polarized second light beam 200b. The ½ wave plates 4a and 4b are an example of a “polarization plane rotator”.

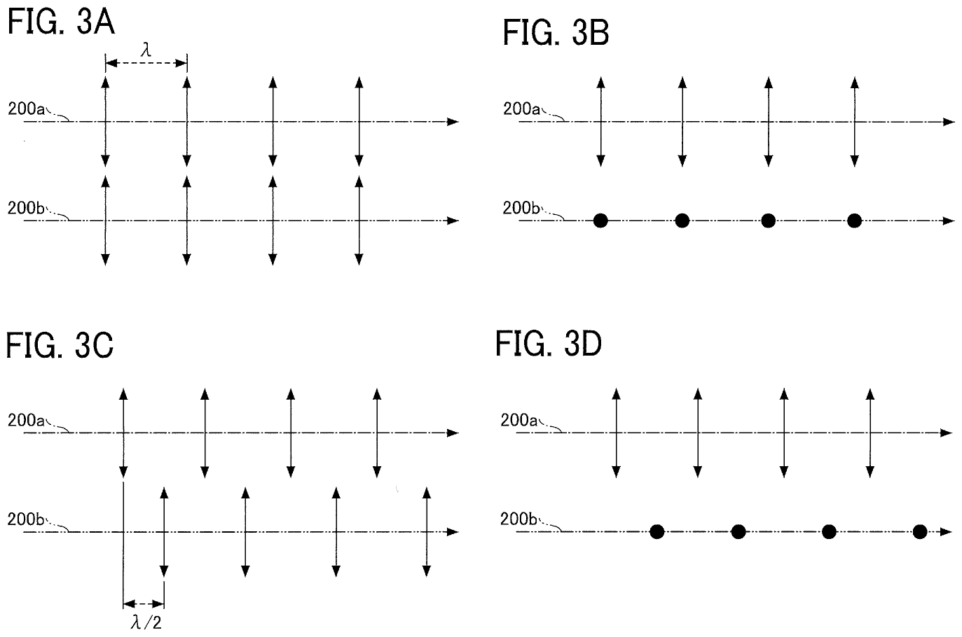

The first light beam 200a and the second light beam 200b incident on the photodetector 6 interfere with each other and an interference fringe pattern is produced in a case where the first light beam 200a and the second light beam 200b have a predetermined relation. FIGS. 3A to 3D are views illustrating relations between the first light beam 200a and the second light beam 200b. The interference fringe pattern is an example of “interfering light”.

In FIGS. 3A to 3D, a chain line arrow represents the first light beam 200a traveling in a direction of the chain line arrow. A two point chain line arrow represents the second light beam 200b traveling in a direction of the two point chain line arrow. Further, as illustrated in FIG. 1, the first light beam 200a and the second light beam 200b travel toward the photodetector 6, and thus traveling directions of the first light beam 200a and the second light beam 200b are not parallel to each other. However, for convenience of explanation, the traveling directions of the first light beam 200a and the second light beam 200b are illustrated as being parallel to each other in FIGS. 3A to 3D.

A solid arrow represents an oscillation direction of the linearly polarized first light beam 200a or the linearly polarized second light beam 200b, which is parallel to a paper plane. In other words, the polarization plane is parallel to the paper plane. A black dot represents an oscillation direction of the linearly polarized first light beam 200a or the linearly polarized second light beam 200b, which is perpendicular to the paper plane. In other words, the polarization plane is perpendicular to the paper plane. A position of the solid arrow or a position of the black dot in the traveling direction of light represents a phase of the light. For example, a length indicated by a dotted arrow in FIG. 3A corresponds to a wavelength λ of the probe light, and a length indicated by a dotted arrow in FIG. 3C corresponds to ½ of the wavelength λ of the probe light.

FIGS. 3A and 3C illustrate a case where the polarization plane of the first light beam 200a and the polarization plane of the second light beam 200b are parallel to each other (the angle α between the polarization plane of the first light beam 200a and the polarization plane of the second light beam 200b is 0 degrees). FIGS. 3B and 3D illustrate a case where the polarization plane of the first light beam 200a and the polarization plane of the second light beam 200b are orthogonal to each other (the angle α between the polarization plane of the first light beam 200a and the polarization plane of the second light beam 200b is 90 degrees).

In FIGS. 3A and 3B, the positions of the solid arrow and the black dot of the first light beam 200a and the second light beam 200b in the traveling direction of the light are aligned with each other. This means that a phase of the first light beam 200a and a phase of the second light beam 200b are aligned with each other. In other words, a phase difference between the first light beam 200a and the second light beam 200b is 0 degrees as a phase angle.

In FIGS. 3C and 3D, the positions of the solid arrow and the black dot of the first light beam 200a and the second light beam 200b in the traveling direction of the light are misaligned with each other by ½ of the wavelength λ. In other words, a phase difference between the first light beam 200a and the second light beam 200b is 180 degrees as a phase angle. The phase angle of 180 degrees may be converted into a length, and the length corresponds to ½ of the wavelength of the probe light.

As illustrated in FIG. 3A, in a case where the polarization plane of the first light beam 200a and the polarization plane of the second light beam 200b are parallel to each other, the first light beam 200a and the second light beam 200b interfere with each other and the interference fringe pattern is produced. Meanwhile, as illustrated in FIG. 3B, in a case where the polarization plane of the first light beam 200a and the polarization plane of the second light beam 200b are orthogonal to each other, the first light beam 200a and the second light beam 200b do not interfere with each other and the interference fringe pattern is not produced. Further, in an intermediate state (0 degrees<α<90 degrees) between the states illustrated in FIGS. 3A and 3B, the first light beam 200a and the second light beam 200b partially interfere with each other and the interference fringe pattern is produced. In other words, only components that have oscillation directions parallel to each other (polarization planes of which are parallel to each other) in linear polarization of the first light beam 200a and the second light beam 200b interfere with each other and the interference fringe pattern is produced.

Such a relation has been known as the Fresnel-Arago laws (for example, see “A law of interference of electromagnetic beams of and state of coherence and polarization and the Fresnel-Arago interference laws”, J. Opt. Soc. Am. A, Vol. 21, No. 12, 2414-2147, December 2004, M. Mujat, A. Dogariu, and E. Wolf).

Similarly, as illustrated in FIG. 3C, in a case where the polarization plane of the first light beam 200a and the polarization plane of the second light beam 200b are parallel to each other, the first light beam 200a and the second light beam 200b interfere with each other and the interference fringe pattern is produced. Meanwhile, as illustrated in FIG. 3D, in a case where the polarization plane of the first light beam 200a and the polarization plane of the second light beam 200b are orthogonal to each other, the first light beam 200a and the second light beam 200b do not interfere with each other and the interference fringe pattern is not produced.

However, in the case illustrated in FIG. 3C, since a phase difference between the first light beam 200a and the second light beam 200b is 180 degrees, destructive interference occurs. As a result, a light intensity of the interference fringe pattern becomes a minimum (significantly low). In the case illustrated in FIG. 3A, since the phase difference between the first light beam 200a and the second light beam 200b is 0 degrees, constructive interference occurs. As a result, a light intensity of the interference fringe pattern becomes a maximum (significantly high). In an intermediate state in which the phase difference is between 0 degrees and 180 degrees, intermediate interference between the constructive interference and the destructive interference occurs, and a light intensity of the interference fringe pattern has a value corresponding to a degree of interference.

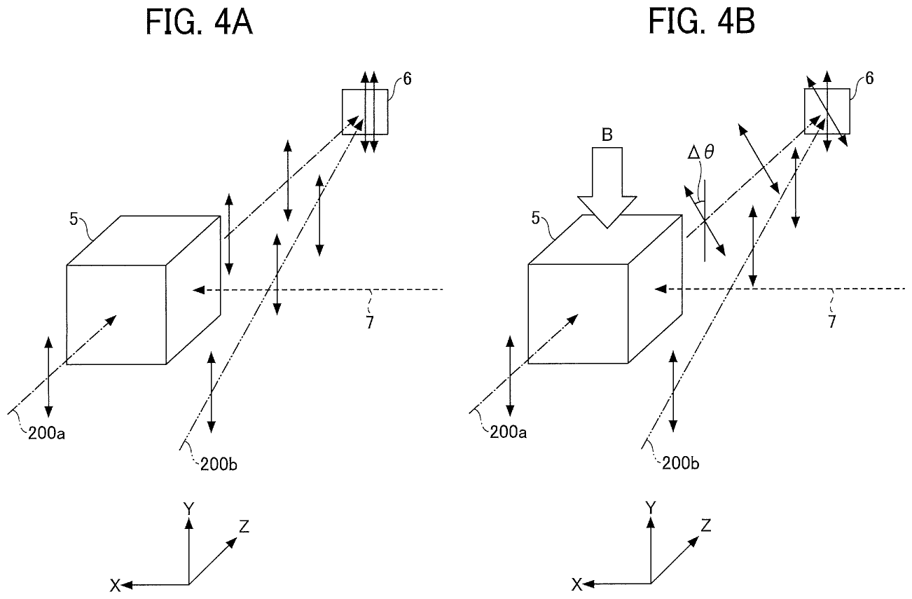

FIGS. 4A and 4B are views for describing behaviors of the first light beam 200a and the second light beam 200b when a magnetic field is applied to the cell 5.

In FIGS. 4A and 4B, a chain line arrow represents the first light beam 200a traveling in a direction of the chain line arrow, and a two point chain line arrow represents the second light beam 200b traveling in a direction of the two point chain line arrow. A solid arrow represents oscillation directions of the linearly polarized first light beam 200a and the linearly polarized second light beam 200b.

In examples illustrated in FIGS. 4A and 4B, the angle α between the polarization plane of the first light beam 200a before passing through the cell 5 and the polarization plane of the second light beam 200b is 0 degrees. The pump light 7 is incident on the cell 5, and the alkali metal atom in the cell 5 is spin-polarized by the optical pumping by the pump light 7.

FIG. 4A illustrates a case where the magnetic field is not applied to the cell 5. The polarized spin of the alkali metal atom in the cell 5 is not rotated and the linearly polarized first light beam 200a whose polarization plane is not changed even after passing through the cell 5, is incident on the photodetector 6. Meanwhile, the linearly polarized second light beam 200b is directly incident on the photodetector 6. The first light beam 200a and the second light beam 200b interfere with each other, and a light intensity I0 of the interference fringe pattern is detected by the photodetector 6.

FIG. 4B illustrates a case where a magnetic field having a strength B is applied to the cell 5. The polarized spin of the alkali metal atom in the cell 5 is rotated according to the strength B of the magnetic field. Due to the Faraday rotation in proportion to the rotation of the polarized spin, the polarization plane of the first light beam 200a incident on the cell 5 is rotated by an angle AO about an axis in the traveling direction of the light. Meanwhile, the linearly polarized second light beam 200b is directly incident on the photodetector 6. The first light beam 200a and the second light beam 200b interfere with each other, and a light intensity I of the interference fringe pattern is detected by the photodetector 6.

A light intensity difference ΔI between the light intensity I0 and the light intensity I is in proportion to the strength B of the magnetic field. Therefore, the strength B of the magnetic field can be measured based on the light intensity difference ΔI detected by the photodetector 6.

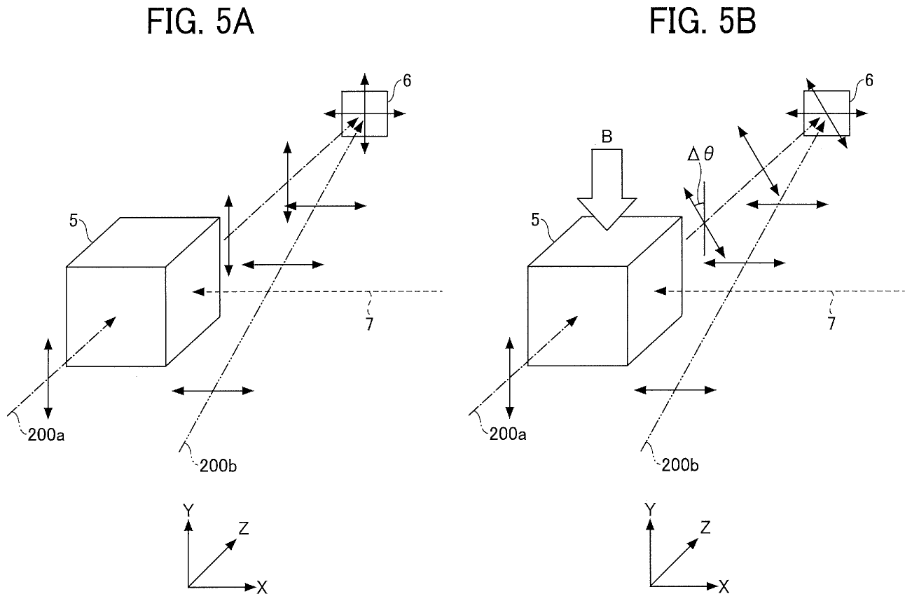

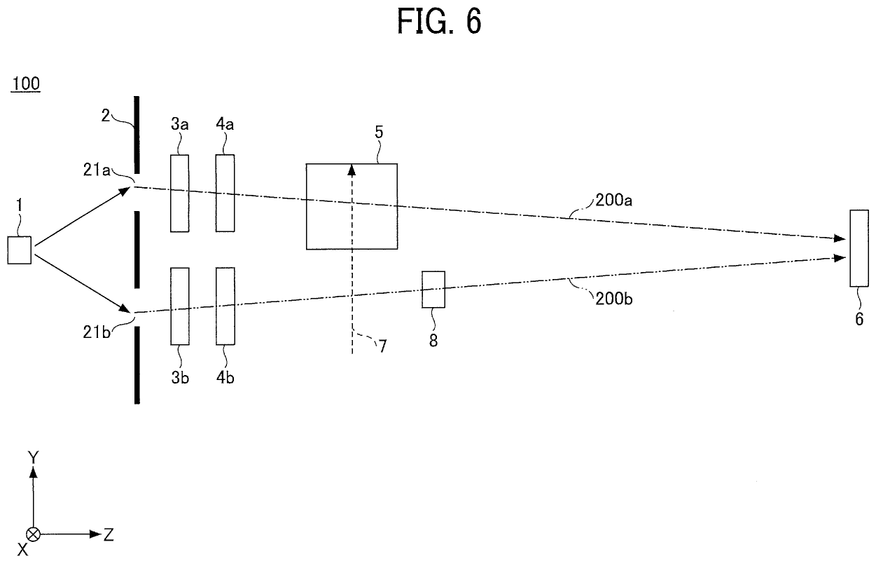

Next, FIGS. 5A and 5B are views for describing behaviors of the first light beam 200a and the second light beam 200b when the magnetic field is applied to the cell 5 in a case where the angle α between the polarization plane of the first light beam 200a before passing through the cell 5 and the polarization plane of the second light beam 200b is 90 degrees.

Examples illustrated in FIGS. 5A and 5B are different from the examples illustrated in FIGS. 4A and 4B only in regard to the polarization plane of the second light beam 200b. Even in this case, the first light beam 200a of which the polarization plane is rotated according to the strength B of the magnetic field and the second light beam 200b interfere with each other, and a light intensity I of the interference fringe pattern is detected by the photodetector 6. However, since the polarization plane of the second light beam 200b is different, the light intensity I of the interference fringe pattern has a value different from the light intensity I of the example illustrated in FIGS. 4A and 4B. The strength B of the magnetic field can be measured based on the light intensity difference ΔI between the light intensity I0 and the light intensity I in a case where the magnetic field is not applied.

Hereinabove, the case where the angle α between the polarization plane of the first light beam 200a and the polarization plane of the second light beam 200b is 0 degrees or 90 degrees has been described. However, the angle α may also be another angle such as 45 degrees.

In the examples illustrated in FIGS. 4A to 5B, the case where the phase difference between the first light beam 200a and the second light beam 200b is 0 degrees has been described. However, the present invention is not limited thereto. The phase difference between the first light beam 200a and the second light beam 200b may be 180 degrees, or may be a predetermined phase difference between 0 degrees and 180 degrees. Such a predetermined phase difference can be applied by, for example, providing a member for setting the phase difference on an optical path along which the first light beam 200a or the second light beam 200b is incident on the photodetector 6.

FIG. 6 is a view for describing a configuration of the atomic magnetometer 100 in a case of applying the predetermined phase difference. In FIG. 6, a phase difference setting member 8 is provided on an optical path along which the second light beam 200b is incident on the photodetector 6. The phase difference setting member 8 is a transparent member made of glass or the like. Since a length of the optical path is changed depending on a refractive index and a thickness (a length of the glass in the traveling direction of the light) of the glass member, predetermined phase delay can be applied to the second light beam 200b to set the phase difference between the first light beam 200a and the second light beam 200b.

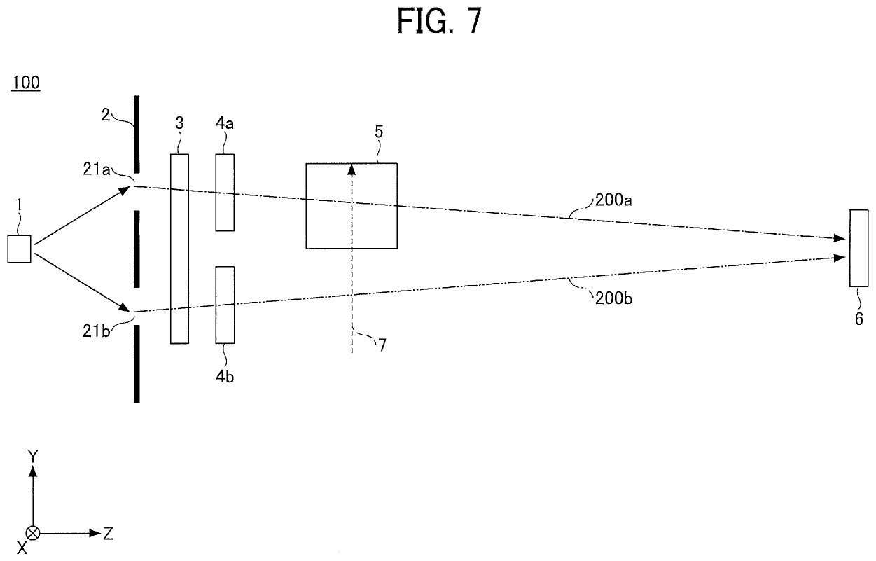

In the example illustrated in FIG. 1, the atomic magnetometer 100 includes two polarizers 3a and 3b. However, the atomic magnetometer 100 may also include only one polarizer. FIG. 7 is a view for describing a configuration of the atomic magnetometer 100 in a case where the atomic magnetometer 100 includes only one polarizer. The atomic magnetometer 100 includes a polarizer 3. Light having passed through the pinholes 21a and 21b passes through the polarizer 3 to have a predetermined linear polarization plane. Alternatively, in a case where linearly polarized light is emitted from the light source 1, the light passes through the polarizer 3a to increase a polarization degree of the linearly polarized light. The configuration of the atomic magnetometer 100 can be simplified by including one polarizer.

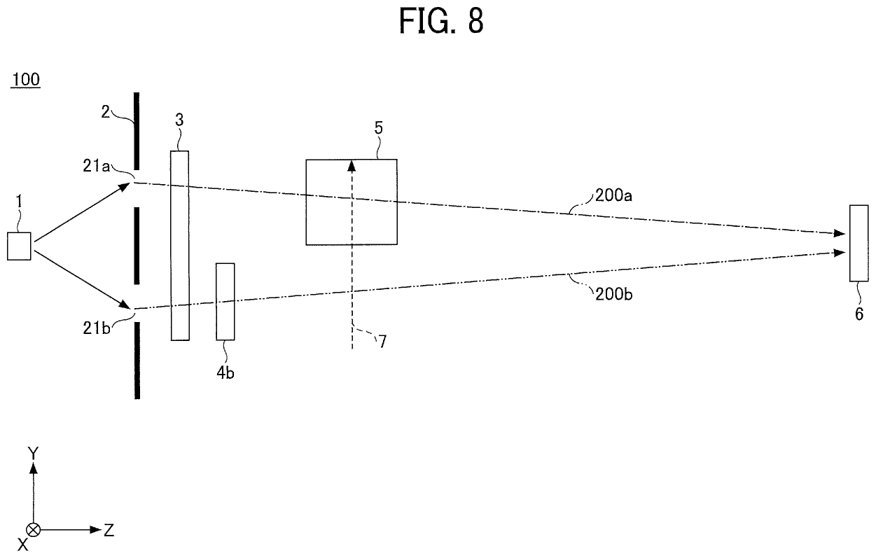

Further, in the example illustrated in FIG. 1, the atomic magnetometer 100 includes two ½ wave plates 4a and 4b. However, the atomic magnetometer 100 may also include one ½ wave plate only on any one of optical paths of light having passed through the pinhole 21a and 21b. FIG. 8 is a view for describing a configuration of the atomic magnetometer 100 in a case where the atomic magnetometer 100 includes only one ½ wave plate. The atomic magnetometer 100 includes the ½ wave plate 4b. An angle of the optical axis of the ½ wave plate 4b can be adjusted to set the angle α between the polarization plane of the first light beam 200a and the polarization plane of the second light beam 200b to a predetermined angle. The configuration of the atomic magnetometer 100 can be simplified by including one ½ wave plate.

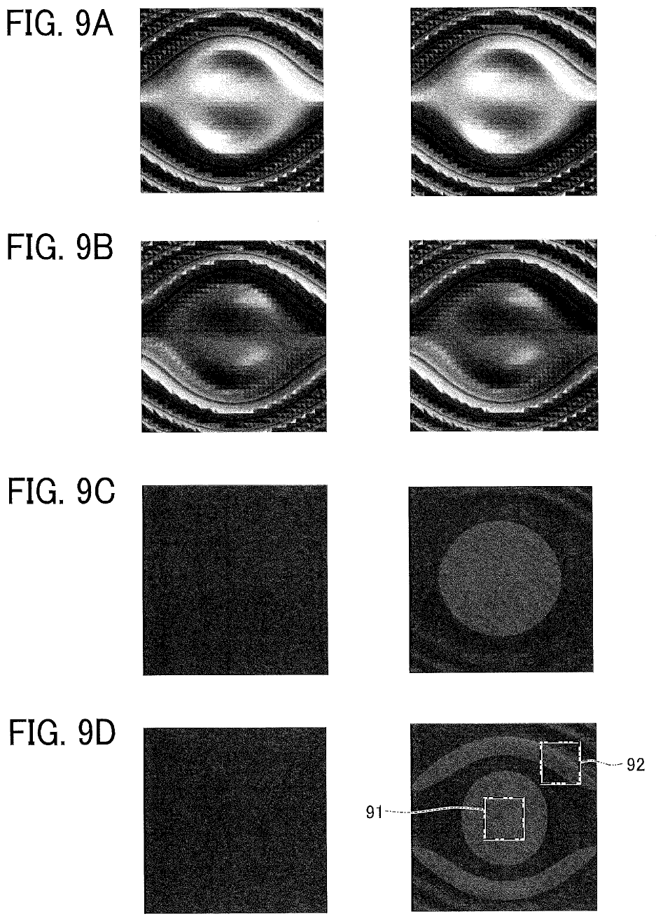

Next, simulation results of the interference fringe pattern detected by the atomic magnetometer 100 according to the present embodiment will be described with reference to FIGS. 9A to 9D. A simulation is performed under the condition that a distance (see FIG. 1 and the like) from the light splitting unit 2 to the photodetector 6 is 50 mm, and a light receiving area of the photodetector 6 is 20×20 (mm²), thereby calculating a distribution of light intensities at a position of the photodetector 6. FIGS. 9A to 9D are views displaying light intensities of the interference fringe pattern which is a simulation result by an effect of light and shade.

In FIGS. 9A to 9D, four views at the left side are views of a case where the magnetic field is not applied. Four views at the right side are views of a case where the magnetic field is applied and the polarization plane of the first light beam 200a is rotated once about an axis in the traveling direction of the light. FIGS. 9A to 9D illustrate results obtained by performing simulations under the four conditions in FIGS. 9A to 9D in the case where the magnetic field is not applied (the left side views) and the case where the magnetic field is applied (the right side views).

The conditions of the simulations are examples and can be arbitrarily changed.

FIG. 9A illustrates a case where the angle α between the polarization plane of the first light beam 200a and the polarization plane of the second light beam 200b is 0 degrees, and the phase difference between the first light beam 200a and the second light beam 200b is 0 degrees. FIG. 9B illustrates a case where the angle α between the polarization plane of the first light beam 200a and the polarization plane of the second light beam 200b is 0 degrees, and the phase difference between the first light beam 200a and the second light beam 200b is 180 degrees. FIG. 9C illustrates a case where the angle α between the polarization plane of the first light beam 200a and the polarization plane of the second light beam 200b is 90 degrees, and the phase difference between the first light beam 200a and the second light beam 200b is 0 degrees. FIG. 9D illustrates a case where the angle α between the polarization plane of the first light beam 200a and the polarization plane of the second light beam 200b is 90 degrees, and the phase difference between the first light beam 200a and the second light beam 200b is 180 degrees.

Light intensities in FIGS. 9A and 9B have light intensity distributions that are the reverse of each other. This is because constructive interference occurs between the first light beam 200a and the second light beam 200b in the case in FIG. 9A, and destructive interference occurs between the first light beam 200a and the second light beam 200b due to the phase difference of 180 degrees in FIG. 9B. In both of FIGS. 9A and 9B, a change in the light intensity distribution (interference fringe pattern) caused by application or non-application of the magnetic field hardly occurs. However, the application or non-application of the magnetic field causes a difference in entire light intensity occurs.

In FIGS. 9C and 9D, as illustrated in the left side views, in a case where the magnetic field is not applied, the interference fringe pattern is not produced and the light intensity distribution is not shown. This is because the angle α between the polarization plane of the first light beam 200a and the polarization plane of the second light beam 200b is 90 degrees and thus the first light beam 200a and the second light beam 200b do not interfere with each other.

As illustrated in the right side views, in a case where the magnetic field is applied, the interference fringe pattern is produced and the light intensity distribution is shown. Further, light intensities in FIGS. 9C and 9D show light intensity distributions that are the reverse of each other. This is because constructive interference occurs between the first light beam 200a and the second light beam 200b in the case in FIG. 9C, and destructive interference occurs between the first light beam 200a and the second light beam 200b due to the phase difference of 180 degrees in FIG. 9D.

In FIGS. 9C and 9D, a change from a state in which the light intensity distribution is not shown to a state in which the light intensity distribution is shown occurs due to the application of the magnetic field, the light intensity distributions (interference fringe pattern) are largely different. Further, with respect to differences between the light intensity distributions caused by the application and non-application of the magnetic field, the differences in FIGS. 9C and 9D are also relatively larger than the differences in FIGS. 9A and 9B.

In the cases in FIGS. 9A to 9D, as a result of quantitatively measuring the light intensity difference ΔI caused by the application and non-application of the magnetic field, it was found that the light intensity difference is the largest in the case in FIG. 9D, the light intensity difference is the next largest in the case in FIG. 9C, and the light intensity differences in the cases in FIGS. 9A and 9B are equivalent to each other. The larger the light intensity difference ΔI caused by the application and non-application of the magnetic field, the more precise the measurement. Therefore, the most precise measurement can be made in the case in FIG. 9D, the next most precise measurement can be made in the case in FIG. 9C, and an equally precise measurement can be made in the cases in FIGS. 9A and 9B.

In FIGS. 9C and 9D, the angle α between the polarization plane of the first light beam 200a and the polarization plane of the second light beam 200b need not be precisely 90 degrees, and a general tolerance such as installation error may be allowed. Similarly, in FIG. 9D, the phase difference need not be precisely 180 degrees, and a general tolerance such as installation error may be allowed.

Next, an example of a setting method of the photodetector 6 according to the present embodiment will be described.

In the present embodiment, for example, calibration is performed in a state in which the magnetic field is not applied before the measurement. In FIG. 9D, for example, the magnetic field is not applied and the state in which the interference fringe pattern is not produced is created. Adjustment for creating the state in which the interference fringe pattern is not produced can be performed by, for example, the ½ wave plate 4a or 4b, or the phase difference setting member 8.

In the state in which the interference fringe pattern is not produced, the light intensity detected by the photodetector 6 is I0. Then, the magnetic field is applied and the light intensity I is detected by the photodetector 6. The light intensity difference ΔI is calculated from the light intensity I and the light intensity JO, and is associated with the strength of the magnetic field. For example, a proportional coefficient of a proportional relation between the light intensity difference ΔI and the strength of the magnetic field is obtained.

Since a region in which the light intensity is relatively high and a region in which the light intensity is relatively low are present according to the light intensity distribution (interference fringe pattern), the light intensity difference ΔI caused by the application and non-application of the magnetic field varies depending on the regions in some cases. For example, in the case in FIG. 9D, the light intensity difference ΔI caused by the application and non-application of the magnetic field in a peripheral region 92 of the photodetector 6 indicated by a two point chain line is larger than the light intensity difference ΔI in a central region 91 of the photodetector 6 indicated by a chain line. As described above, the larger the light intensity difference ΔI caused by the application and non-application of the magnetic field, the more precise the measurement. Therefore, in this case, the region 92 may be used as a light intensity detection region.

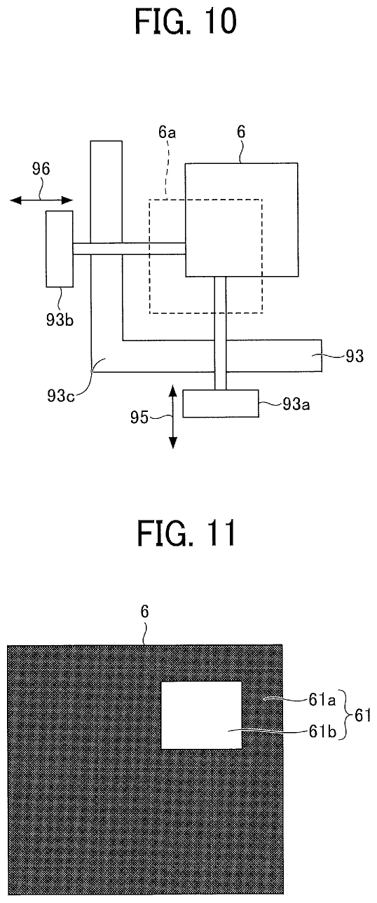

For example, a position of the photodetector 6 may be adjusted so that the photodetector 6 is disposed in the region 92. FIG. 10 is a view illustrating an example of a configuration for adjusting the position of the photodetector 6. The atomic magnetometer 100 includes a position adjusting unit (adjustor) 93 which, for example, moves the photodetector 6 from a position (photodetector 6a) indicated by a dotted line to a position (photodetector 6) indicated by a solid line.

The position adjusting unit 93 includes micrometer heads 93a and 93b, and a supporting member 93c supporting the micrometer heads 93a and 93b so that the micrometer heads 93a and 93b can advance and retreat. The photodetector 6 can advance and retreat in a direction of an arrow 95 by the micrometer head 93a and a spring (not illustrated), and the photodetector 6 can advance and retreat in a direction of an arrow 96 by the micrometer head 93b and a spring. The photodetector 6 can be moved to the region 92 in which the light intensity difference ΔI is large by the position adjusting unit 93, thereby measuring the strength of the magnetic field with high precision. The configuration illustrated in FIG. 10 is suitable, for example, for a case where a light receiving area of the photodetector 6 actually used in the atomic magnetometer 100 is smaller than the light receiving area of 20×20 (mm²) assumed in the simulation.

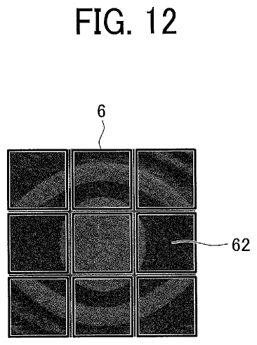

In addition, for example, an opening for passing the light only in the region 92 in which the light intensity difference ΔI is large, and shielding the light in other regions may be provided. FIG. 11 is a view illustrating a configuration of an opening included in the atomic magnetometer 100. In FIG. 11, a mask member 61 is installed on a light receiving surface of the photodetector 6. The mask member 61 includes an opening portion 61a and a shielding portion 61b. The opening portion 61a passes the light and the shielding portion 61b shields the light. With this arrangement, reception of light by the photodetector 6 is possible only in a region of the opening portion 61a. The mask member 61 allows reception of light by the photodetector 6 only in the region 92 in which the light intensity difference ΔI is large, such that the photodetector 6 can measure the strength of the magnetic field with high precision. The configuration illustrated in FIG. 11 is suitable, for example, for a case where a light receiving area of the photodetector 6 actually used in the atomic magnetometer 100 is larger than the light receiving area of 20×20 (mm²) assumed in the simulation.

Further, as illustrated in FIG. 12, a photodetector array 6 including a plurality of pixels 62 may be used to select and extract an output of a pixel corresponding to the region 92 in which the light intensity difference ΔI is large, thereby increasing precision of measurement. Examples of the photodetector array 6 including the plurality of pixels 62 include a photodiode array, an imaging device such as a CMOS or a CCD, or the like.

As described above, in the present embodiment, the atomic magnetometer 100 includes the photodetector 6 which receives the light which has passed through the cell 5 and the light which has not passed through the cell 5, among the two light beams which have been split by the light splitting unit 2. Since detection of a signal for magnetic field measurement is performed by only one photodetector 6, noise mixed into the photodetector can be suppressed, in comparison to a case where a plurality of photodetectors is used. Further, degradation of precision of magnetic field measurement due to the noise mixed into the photodetector can be suppressed.

In addition, the strength B of the magnetic field is obtained by multiplying the intensity of the light detected by the photodetector 6 by the proportional coefficient, or the like, and thus a processing of a detection signal using an electric and electronic circuit is not required. This reduces degradation of precision of the magnetic field measurement due to noise mixed into the electric and electronic circuit.

In the present embodiment, precision of the magnetic field measurement performed by the atomic magnetometer 100 is improved as described above.

### Second Embodiment

Next, an atomic magnetometer 100a according to a second embodiment will be described. An overlapping description of the same components as the components already described in the first embodiment may be omitted.

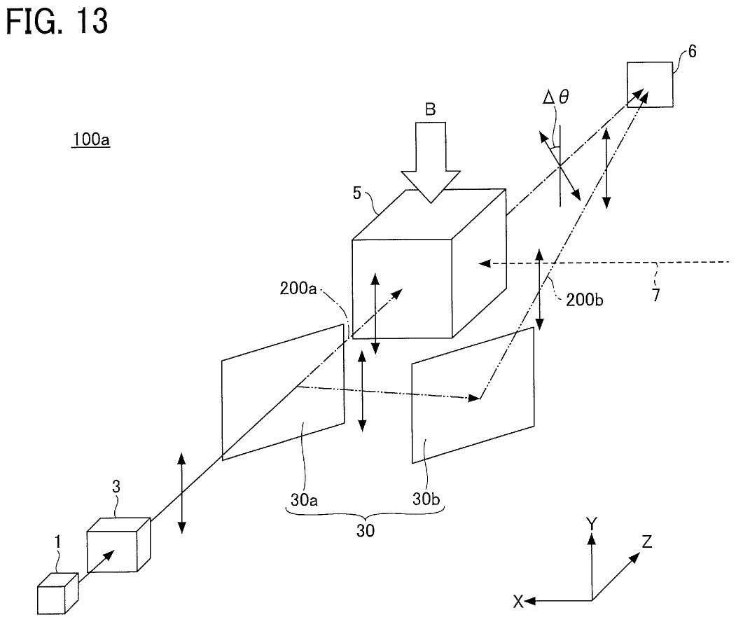

FIG. 13 is a view for describing a configuration of the atomic magnetometer 100a according to the present embodiment.

As illustrated in FIG. 13, the atomic magnetometer 100a includes a light splitting unit 30. The light splitting unit 30 includes a half mirror 30a and a mirror 30b.

The half mirror 30a reflects a part of light having emitted from a light source 1 and having passed through a polarizer 3 and transmits the remaining light. The half mirror 30a is configured so that a ratio of an intensity of the reflected light to an intensity of the transmitted light is 1:1. However, the ratio of the intensity of the reflected light to the intensity of the transmitted light is not limited thereto, but may be arbitrarily set.

The light having transmitted through the half mirror 30a passes through a cell 5 and then is incident on the photodetector 6. The light having transmitted through the half mirror 30a is first light beam 200a. Meanwhile, the light reflected by the half mirror 30a is deflected by the mirror 30b to be incident on a photodetector 6. The light reflected by the half mirror 30a is second light beam 200b. Further, the mirror 30b is an example of a “deflector”.

In the example illustrated in FIG. 13, the atomic magnetometer 100a does not include a ½ wave plate. However, the atomic magnetometer 100a may include the ½ wave plate. In this case, the ½ wave plate may be disposed on an optical path between the half mirror 30a and the mirror 30b, or an optical path from the mirror 30b to the photodetector 6. With this arrangement, it is possible to rotate a polarization plane of the second light beam 200b by any angle such as 90 degrees.

The ½ wave plate may also be disposed on an optical path between the half mirror 30a and the cell 5. With this arrangement, it is possible to rotate a polarization plane of the first light beam 200a by any angle.

Instead of the half mirror 30a, a beam splitter such as a cube beam splitter may be used. Further, instead of the mirror 30b, a reflective prism or the like may be used, or any combination of the mirror 30b and the reflective prism may be used.

In the present embodiment, the light splitting unit 30 includes the half mirror 30a and the mirror 30b. Arrangement of the half mirror 30a and the mirror 30b can be adjusted to flexibly set optical paths of the first light beam 200a and the second light beam 200b. For example, the arrangement of the half mirror 30a and the mirror 30b can be adjusted to miniaturize the atomic magnetometer 100a. As the half mirror or the beam splitter is used as the light splitting unit, it is possible to increase an amount of probe light in comparison to a case where a pinhole or a slit is used as the light splitting unit, or the like, thereby improving precision of measurement.

Effects other than the effects described above are the same as the effects described in the first embodiment.

### Third Embodiment

Next, an atomic magnetometer 100b according to a third embodiment will be described. An overlapping description of the same components as the components already described in the first and second embodiments may be omitted.

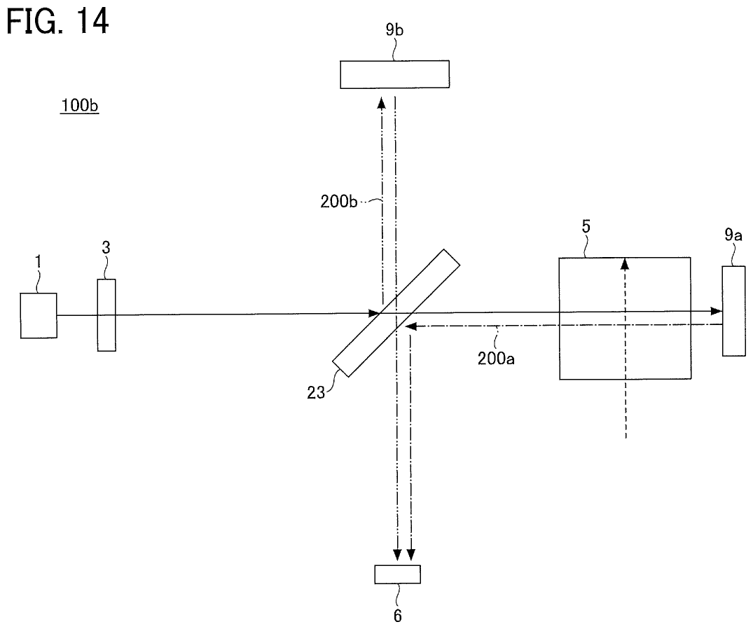

FIG. 14 is a view for describing a configuration of the atomic magnetometer 100b according to the present embodiment.

As illustrated in FIG. 14, the atomic magnetometer 100b includes a light splitting unit 23, a mirror 9a, and a mirror 9b. Examples of the light splitting unit 23 include a half mirror. The light splitting unit 23 reflects a part of light having emitted from a light source 1 and having passed through a polarizer 3 and transmits the remaining light.

The light reflected by the light splitting unit 23 is deflected by the mirror 9b to be turned back in a reverse direction, transmits through the light splitting unit 23, and is incident on a photodetector 6. The light deflected by the mirror 9b and incident on the photodetector 6 is second light beam 200b. Further, the mirror 9b is an example of a “deflector”.

Meanwhile, the light having transmitted through the light splitting unit 23 passes through the cell 5 and is reflected by the mirror 9a to be turned back in a reverse direction. The light reflected by the mirror 9a is reflected by the light splitting unit 23 toward the photodetector 6 and is incident on the photodetector 6. The light reflected by the mirror 9a and incident on the photodetector 6 is first light beam 200a.

An angle of reflection by the light splitting unit 23 is not limited to 90 degrees as illustrated in FIG. 14, but may be any angle. In this case, the mirror 9a is installed so as to reflect the light incident on the mirror 9a in a reverse direction at an angle parallel to the incident light. Similarly, the mirror 9b is also installed so as to reflect the light incident on the mirror 9b in a reverse direction at an angle parallel to the incident light.

Similarly to the second embodiment, a ½ wave plate may be installed on any optical path. A half mirror may be used instead of the light splitting unit, or a reflective prism may be used instead of at least one of the mirror 9a and the mirror 9b.

In the present embodiment, the atomic magnetometer 100b includes the light splitting unit 23, the mirror 9a, and the mirror 9b. Arrangement of the light splitting unit 23, the mirror 9a, and the mirror 9b can be adjusted to flexibly set optical paths of the first light beam 200a and the second light beam 200b. For example, the arrangement of the light splitting unit 23, the mirror 9a, and the mirror 9b can be adjusted to miniaturize the atomic magnetometer 100b. Further, the first light beam 200a passes the cell 5 twice in a reciprocating manner and thus the first light beam 200a can be doubly affected by the magnetic field applied to the cell 5 in comparison to a case where the first light beam 200a passes the cell 5 once. As a result, a light intensity difference ΔI can be increased, and precision of measurement can be improved.

Effects other than the effects described above are the same as the effects described in the first embodiment.

### Fourth Embodiment

Next, a gradiometer according to a fourth embodiment will be described. An overlapping description of the same components as the components already described in the first to third embodiments may be omitted.

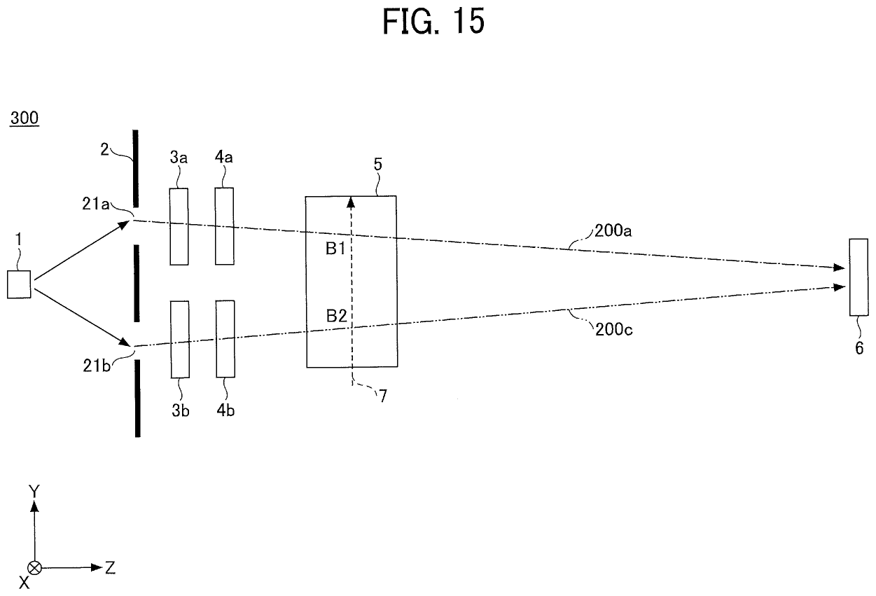

The gradiometer according to the present embodiment includes the atomic magnetometer 100 according to the first embodiment, or the like. FIG. 15 is a view for describing a configuration of a gradiometer 300 according to the present embodiment. The gradiometer 300 according to the present embodiment is different from the atomic magnetometer 100 according to the first embodiment in that light having been split by a light splitting unit 2 and having passed through a pinhole 21b passes through a cell 5 and then is incident on a photodetector 6. The light having passed through the pinhole 21b and the cell in this order, and then incident on the photodetector 6 is third light 200c.

The photodetector 6 detects a light intensity of an interference fringe pattern caused by interference between first light beam 200a and the third light 200c.

A magnetic field having a strength B1 and a magnetic field having a strength B2 are applied to the cell 5. The strength B1 and the strength B2 are different from each other. The light intensity detected by the photodetector 6 is changed depending on the magnetic field having the strength B1 and the magnetic field having the strength B2. The gradiometer 300 detects such a light intensity, and thus can measure an intensity difference between the magnetic field having the strength B1 and the magnetic field having the strength B2, that is, the magnetic field gradient. The light intensity and the magnetic gradient are associated with each other in advance in simulation or the like. For example, in a case where the light intensity and the magnetic gradient are in proportion to each other, a proportional coefficient is obtained in advance and the proportional coefficient is multiplied to the light intensity detected by the photodetector 6, thereby calculating the magnetic field gradient.

As described above, the gradiometer according to the present embodiment can be implemented. The present embodiment exhibits the same effects as the effects of the atomic magnetometer according to the first embodiment. Further, the configurations of the atomic magnetometers 100a and 100b according to the second and third embodiments can also be applied to the gradiometer. In this case, the same effects as the effects described in the second and third embodiments can be obtained.

### Fifth Embodiment

Next, a biomagnetism measurement apparatus according to a fifth embodiment will be described. An overlapping description of the same components as the components already described in the first to fourth embodiments may be omitted.

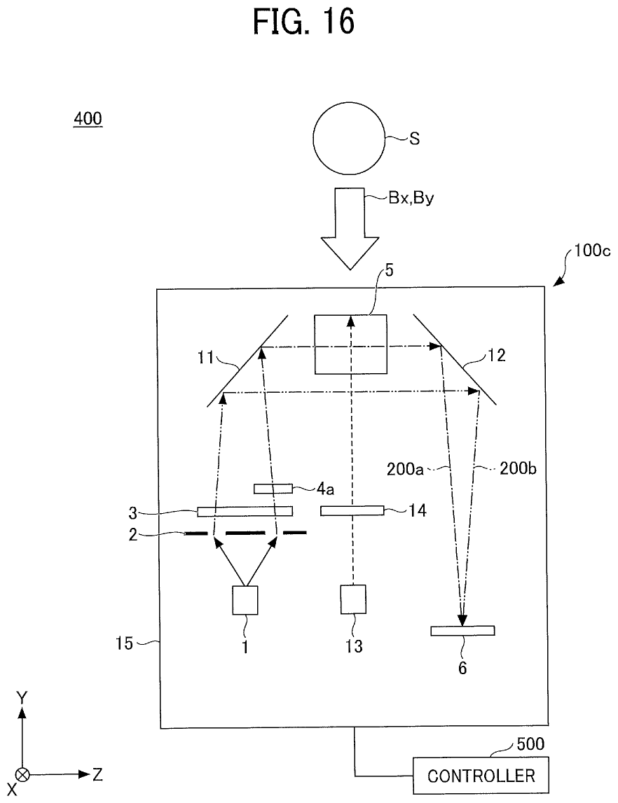

FIG. 16 is a view for describing a configuration of a biomagnetism measurement apparatus 400 according to the present embodiment.

The biomagnetism measurement apparatus 400 includes an atomic magnetometer 100c and a controller 500, and measures a strength of Bx of an X-direction component and a strength By of a Y-direction component in a strength B applied to a measurement target S. A magnetic field having the strength Bx is a magnetic field in the X direction indicated by an arrow in FIG. 16, and a magnetic field having the strength By is a magnetic field in the Y direction.

The strength B generated from the measurement target S is Bx+By+Bz (B=Bx+By+Bz) when being represented by vectors in the X direction, the Y direction, and the Z direction, and a magnitude PI of the magnetic field is (Bx2+By2+Bz2)^{1/2} (|B|=(Bx2+By2+Bz2)^{1/2}). In a case where the atomic magnetometer 100c is disposed as illustrated in FIG. 16, a magnetic field in the X direction and the Y direction is measured. This is because the atomic magnetometer 100c has no sensitivity in the Z direction which is a traveling direction of probe light.

In a case where a human is a target, a magnetic field generated from the brain, the heart, bone marrow, or the like becomes the measurement target S. At the time of measurement, for example, the atomic magnetometer 100c is brought close to the measurement target S and is disposed so that the magnetic field generated from the measurement target S is applied to a cell 5. Further, at the time of measurement, the cell 5 is required to be heated by a heating unit (not illustrated), and heat applied to the cell 5 is insulated from a case.

The atomic magnetometer 100c includes a mirror 11, a mirror 12, a pump-specific light source 13, and a ¼ wave plate 14. These components and other components such as a light source 1 and the like are disposed in a case 15 together.

The mirror 11 reflects light having emitted from the light source 1 and having passed through a light splitting unit 2, a polarizer 3, a ½ wave plate 4a, and the like, in a positive Z direction as illustrated in FIG. 16. The mirror 12 reflects the light reflected by the mirror 11 toward a photodetector 6 in the negative Y direction.

The pump-specific light source 13 emits light having an absorption wavelength (for example, 895 nm corresponding to a D1 line of 133 Cs) of an alkali metal atom in the cell 5. Examples of the pump-specific light source 13 include a VCSEL. However, as long as the pump-specific light source 13 may emit the light having the absorption wavelength of the alkali metal atom, the pump-specific light source 13 is not limited to the VCSEL.

The ¼ wave plate 14 converts linearly polarized light emitted from the pump-specific light source 13 into nearly circularly polarized light and irradiates the cell 5 with the nearly circularly polarized light. As described above, the alkali metal atom is excited by the nearly circularly polarized light, thereby making it possible to increase a pumping rate.

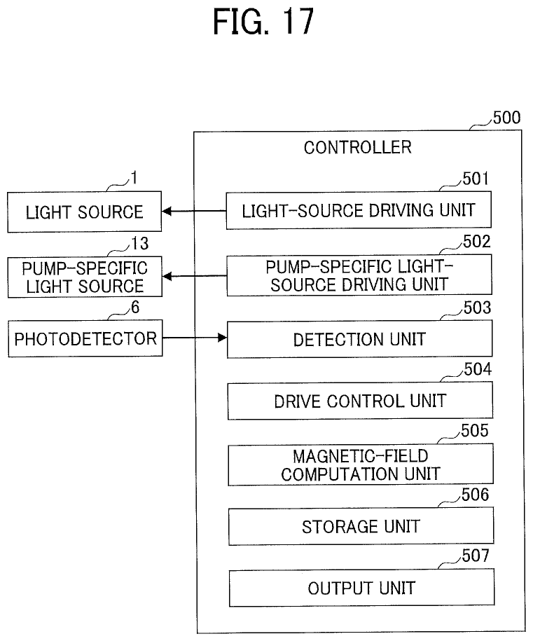

FIG. 17 is a functional block diagram illustrating components of the controller 500 according to the present embodiment. The respective functional blocks illustrated in FIG. 17 are conceptual and are not necessarily be physically configured as illustrated. A part or all of the functional blocks can be functionally or physically distributed or combined in any unit. A part or all of processing functions performed by the respective functional block can be implemented by a program executed by a control processing unit (CPU) or can be implemented as hardware by wired logic.

The controller 500 includes a light-source driving unit 501, a pump-specific light-source driving unit 502, a detection unit 503, a drive control unit 504, a magnetic-field computation unit 505, a storage unit 506, and an output unit 507.

The light-source driving unit 501 is electrically coupled to the light source 1 by a cable or the like, controls turning-on or turning-off of the light source 1, and controls an intensity of light emitted from the light source 1. The light-source driving unit 501 is implemented by, for example, an electric circuit which applies a driving voltage to the light source 1 based on a control signal.

The pump-specific light-source driving unit 502 is electrically coupled to the pump-specific light source 13 by a cable or the like, controls turning-on or turning-off of the pump-specific light source 13, and controls an intensity of light emitted from the pump-specific light source 13. The pump-specific light-source driving unit 502 is implemented by, for example, an electric circuit which applies a driving voltage to the pump-specific light source 13 based on a control signal.

The detection unit 503 is electrically coupled to the photodetector 6, inputs a detection signal from the photodetector 6, and outputs the detection signal to the magnetic-field computation unit 505 or the storage unit 506. The detection unit 503 is implemented by, for example, an analog/digital (A/D) conversion circuit which converts the detection signal from the photodetector 6 from an analog voltage signal into a digital voltage signal.

The drive control unit 504 outputs a control signal to the light-source driving unit 501 or the pump-specific light-source driving unit 502. The magnetic-field computation unit 505 calculates a magnetic field of a measurement target S based on the detection signal of the detection unit 503. The drive control unit 504 and the magnetic-field computation unit 505 are implemented, for example, in a manner that a CPU executes a program stored in a read only memory (ROM) or the like with a random access memory (RAM) as a work area.

The storage unit 506 stores a calculation result obtained by the magnetic-field computation unit 505 and stores a setting value such as a proportional coefficient for calculating the magnetic field from the detection signal of the detection unit 503. The storage unit 506 is implemented by a hard disk drive (HDD), a non-volatile memory (NVRAM), or the like.

The output unit 507 is an interface (I/F) which outputs the calculation result obtained by the magnetic-field computation unit 505 to an external apparatus. Examples of the external apparatus include a personal computer (PC) and the like.

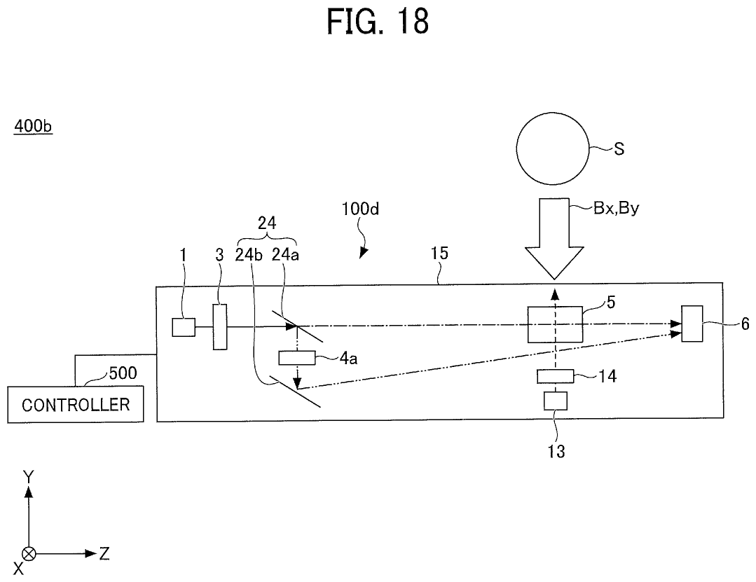

FIG. 18 is a view illustrating a configuration of a biomagnetism measurement apparatus 400b according to a first modified example. The biomagnetism measurement apparatus 400b includes a light splitting unit 24, and the light splitting unit 24 includes a beam splitter 24a and a mirror 24b. The beam splitter 24a reflects a part of light having emitted from a light source 1 and having passed through a polarizer 3 and transmits the remaining light. The light reflected by the beam splitter 24a is second light deflected by the mirror 24b and incident on a photodetector 6. Meanwhile, the light having transmitted through the beam splitter 24a is first light passing through a cell 5 and incident on the photodetector 6. Further, the mirror 24b is an example of a “deflector”.

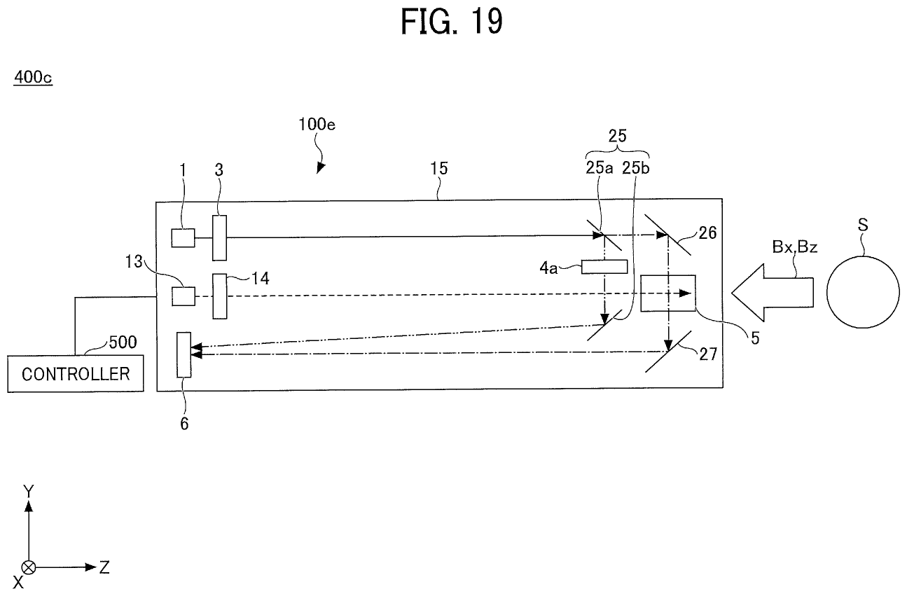

FIG. 19 is a view illustrating a configuration of a biomagnetism measurement apparatus 400c according to a second modified example. The biomagnetism measurement apparatus 400c includes a light splitting unit 25, and the light splitting unit 25 includes a beam splitter 25a and a mirror 25b. The beam splitter 25a reflects a part of light having emitted from a light source 1 and having passed through a polarizer 3 and transmits the remaining light.

The light reflected by the beam splitter 25a is second light deflected by the mirror 25b and incident on a photodetector 6. Meanwhile, the light having transmitted through the beam splitter 25a is reflected by the mirror 26 and transmits through a cell 5. The light having transmitted through the cell 5 is first light reflected by the mirror 27 and incident on the photodetector 6. Further, the mirror 25b is an example of a “deflector”.

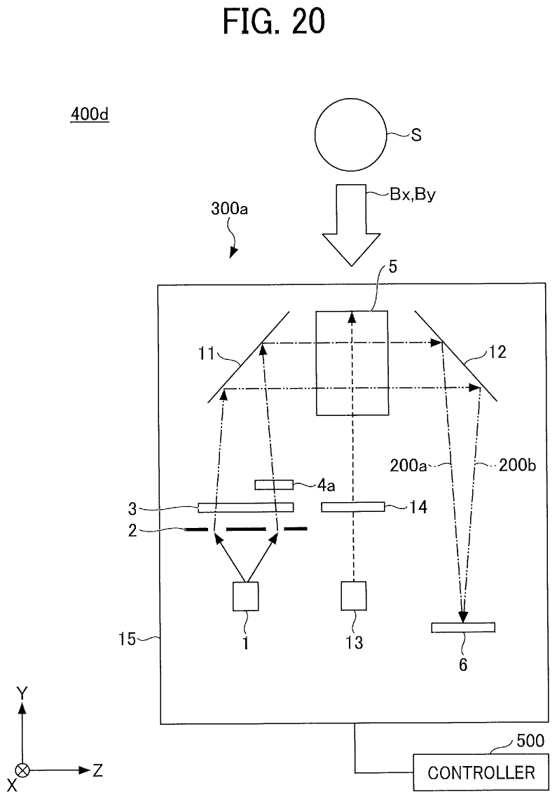

FIG. 20 is a view illustrating a configuration of a biomagnetism measurement apparatus 400d according to a third modified example. The biomagnetism measurement apparatus 400d includes a gradiometer 300a. The gradiometer 300a is different from the atomic magnetometer 100c according to the fifth embodiment in that both of the two light beams which have been split by a light splitting unit 2 pass through a cell 5.

As described above, the biomagnetism measurement apparatus can be implemented by the present embodiment. The present embodiment exhibits the same effects as the effects of the atomic magnetometer 100 according to the first embodiment.

In the present embodiment, as illustrated in FIG. 16, light having emitted from a light source 1 and having passed through the light splitting unit 2 and the like is reflected by a mirror 11, and the light reflected by the mirror 11 is reflected by a mirror 12 toward a photodetector 6. With this arrangement, the atomic magnetometer 100c can be miniaturized and may be easily brought close to a measurement target S, and the like.

The atomic magnetometers 100a and 100b according to the second and third embodiments can also be applied to the biomagnetism measurement apparatus. In this case, the same effects as the effects described in the second and third embodiments can be obtained.

The above-described embodiments are illustrative and do not limit the present invention. Thus, numerous additional modifications and variations are possible in light of the above teachings. For example, elements and/or features of different illustrative embodiments may be combined with each other and/or substituted for each other within the scope of the present invention.

Each of the functions of the described embodiments, performed by the controller, may be implemented by one or more processing circuits or circuitry. Processing circuitry includes a programmed processor, as a processor includes circuitry. A processing circuit also includes devices such as an application specific integrated circuit (ASIC), digital signal processor (DSP), field programmable gate array (FPGA), and conventional circuit components arranged to perform the recited functions.

1. An atomic magnetometer comprising:
a laser light source configured to emit light;
a light splitting unit configured to split the light emitted from the laser light source into at least a first light beam and a second light beam;
a transparent cell filled with an alkali metal atom and through which the first light beam is transmitted; and
a photodetector configured to receive the first light beam which has transmitted through the transparent cell and the second light beam which has not transmitted through the transparent cell.

2. The atomic magnetometer according to claim 1, further comprising a position adjustor configured to adjust a position of the photodetector.

3. The atomic magnetometer according to claim 1,
wherein the alkali metal atom is any one of potassium, rubidium, and cesium.

4. A gradiometer comprising the atomic magnetometer according to claim 1.

5. A biomagnetism measurement apparatus comprising:
the atomic magnetometer according to claim 1; and
a controller configured to control the atomic magnetometer.

6. An atomic magnetometer for measuring a strength of a magnetic field using probe light, the atomic magnetometer comprising:
a laser light source configured to emit probe light;
a light splitting unit configured to split the probe light into a first light beam and a second light beam;
a transparent cell filled with an alkali metal atom and through which the first light beam is transmitted; and
a photodetector configured to detect an intensity of interfering light between the first light beam having transmitted through the transparent cell and the second light beam.

7. The atomic magnetometer according to claim 6,
wherein the first light beam and the second light beam are each a linearly polarized light beam, and
the atomic magnetometer further comprises a polarization plane rotator configured to rotate a polarization plane of at least one of the first light beam and the second light beam.

8. The atomic magnetometer according to claim 7,
wherein the polarization plane of the first light beam is not parallel to the polarization plane of the second light beam.

9. The atomic magnetometer according to claim 7,
wherein the polarization plane of the first light beam is orthogonal to the polarization plane of the second light beam.

10. The atomic magnetometer according to claim 6,
wherein a phase difference between the first light beam and the second light beam is 180 degrees.

11. The atomic magnetometer according to claim 6, further comprising a position adjustor configured to adjust a position of the photodetector.

12. The atomic magnetometer according to claim 6, further comprising a deflector configured to deflect the second light beam toward the photodetector.

13. The atomic magnetometer according to claim 6,
wherein the alkali metal atom is any one of potassium, rubidium, and cesium.

14. A gradiometer comprising the atomic magnetometer according to claim 6.

15. A biomagnetism measurement apparatus comprising:
the atomic magnetometer according to claim 6; and
a controller configured to control the atomic magnetometer.
